(19) United States
(12) Patent Application Publication
(10) Pub. No.: US 2002/0002324 A1
(43) Pub. Date: Jan. 3, 2002

(54) Device for use in surgery

(76) Inventor: Ronan Bernard McManus, Bray (IE)

Correspondence Address: Finnegan, Henderson, Farabow,, Garrett & Dunner, L.L.P., 1300 I Street, N.W., Washington, DC, 20005-3315, US

(21) Appl. No.: 09/870,647

(22) Filed: Jun. 1, 2001

Related U.S. Application Data
(63) Continuation-in-part of application No. 09/801,826, filed on Mar. 9, 2001.
Application No. 09/801,826 is a continuation of application No. PCT/IE99/00122, filed on Dec. 1, 1999.

(30) Foreign Application Priority Data
Dec. 1, 1998 (IE) 980997
Feb. 15, 1999 (IE) 990111
Jun. 1, 2000 (IE) 2000/0444

(51) Int. Cl. A61B 17/02

(52) U.S. Cl. 600/208; 600/233

(57) ABSTRACT

In one embodiment, a wound retractor includes a plurality of straps connected between an inner ring and an outer ring assembly. The inner ring is positioned internal of the wound so that it rests against the innermost tissue layer, and the outer ring is located outside of the wound adjacent the outermost tissue layer. The outer ring assembly includes an inner ring having guide holes for the passage of the straps and an outer ring to which the straps are affixed. Rotation of the outer ring of the outer ring assembly relative to the inner ring of the outer ring assembly results in a shorting of the effective length of the straps, retraction of the wound edge and exposure of the wound site.

### INTRODUCTION

The invention relates to a surgical wound retractor device.

Various retractors are known. However in general known retractors are difficult and cumbersome to use, and/or relatively expensive and/or require sterilisation. This invention is direct towards providing an improved wound retractor which will over come at least some of these problems.

### STATEMENTS OF INVENTION

According to the invention there is provided a surgical wound retractor comprising:an

an inner mounting means for insertion into a wound opening to be retained inside the wound opening;

an outer retracting means;

connecting means between the inner mounting means and the outer retracting means;

the outer retracting means being operable to pull the connecting means laterally to retract the wound opening.

In one embodiment of the invention the connecting means comprises a plurality of spaced-apart straps extending between the inner mounting means and the outer retracting means.

Preferably the straps are of pliable material. Ideally, the straps are of polymeric material.

In a preferred embodiment of the invention the outer retracting means comprises at least two elements, the connecting means being mounted to one of the elements and the elements being movable relative to each other to tension the connecting means. Preferably the retracting means is a ring assembly comprising a first ring and a second ring, the connector means being mounted to one of the rings and one of the rings being movable relative to the other ring to tension the connecting means. Ideally the ring assembly comprises an inner ring and an outer ring, the connector means comprising a plurality of straps, each strap being mounted to the outer ring, and the outer ring being rotatable relative to the inner ring to tension the straps. Preferably the straps extend through guides in the inner ring.

In a preferred embodiment the inner mounting means is an O-ring.

Preferably the ring assembly has a larger diameter than the diameter of the inner ring means.

In one embodiment the inner O-ring is of a flexible material.

### BRIEF DESCRIPTION OF THE DRAWINGS

The invention will be more clearly understood from the following description thereof given by way of example only with reference to the accompanying drawings, in which:

FIG. 1 is a perspective, partially cut-away view of a retractor device according to the invention in a relaxed configuration;

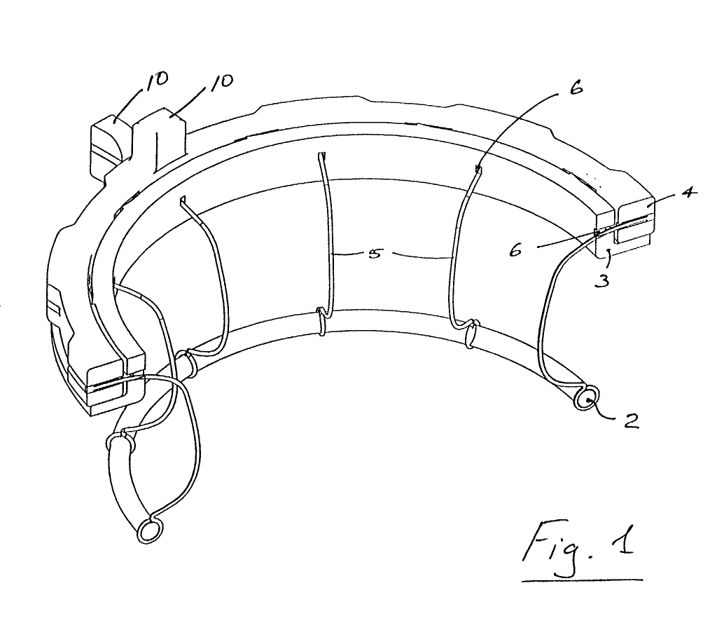

FIG. 2 is a perspective view, from above, of the device of FIG. 1 in a wound opening;

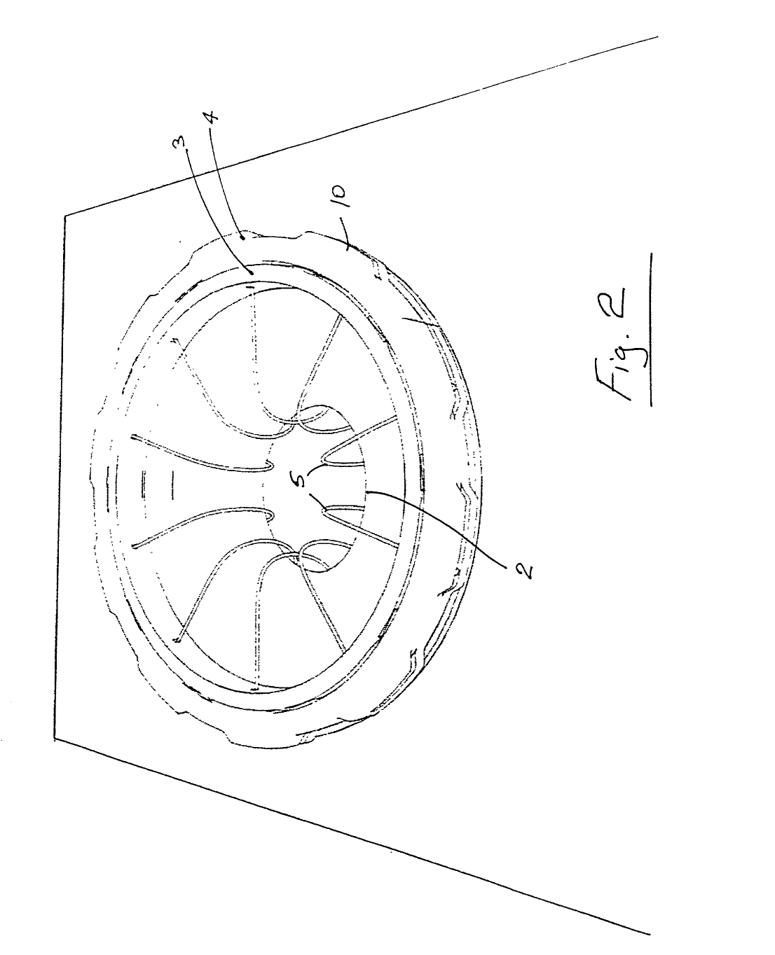

FIG. 3 is a perspective, partially cut-away view of the retractor device of FIG. 2 in a tensioned configuration;

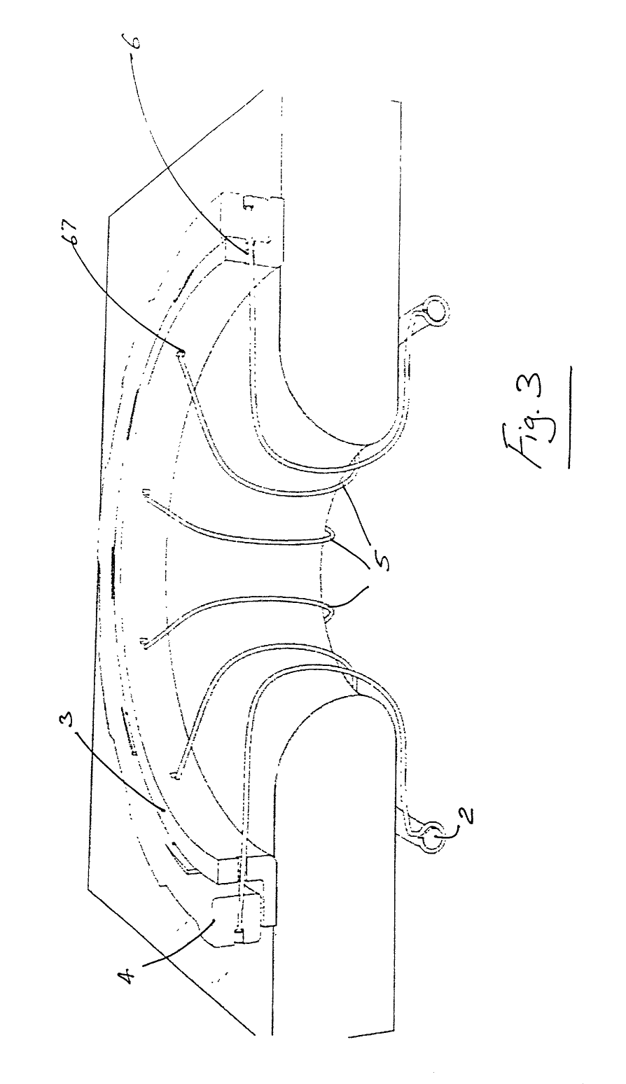

FIG. 4 is a perspective view, from above, of the device of FIG. 3, in a retracted configuration; and

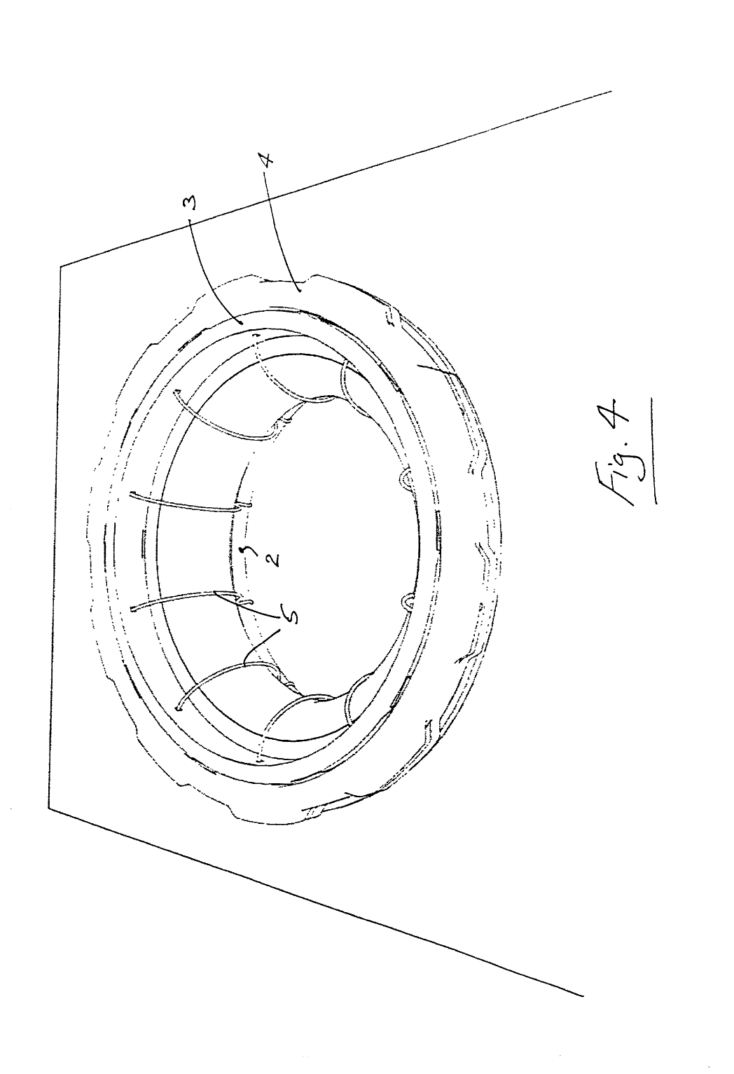

FIG. 5 is a perspective, partially cut-away view of the retractor device in the retracted configuration of FIG. 4.

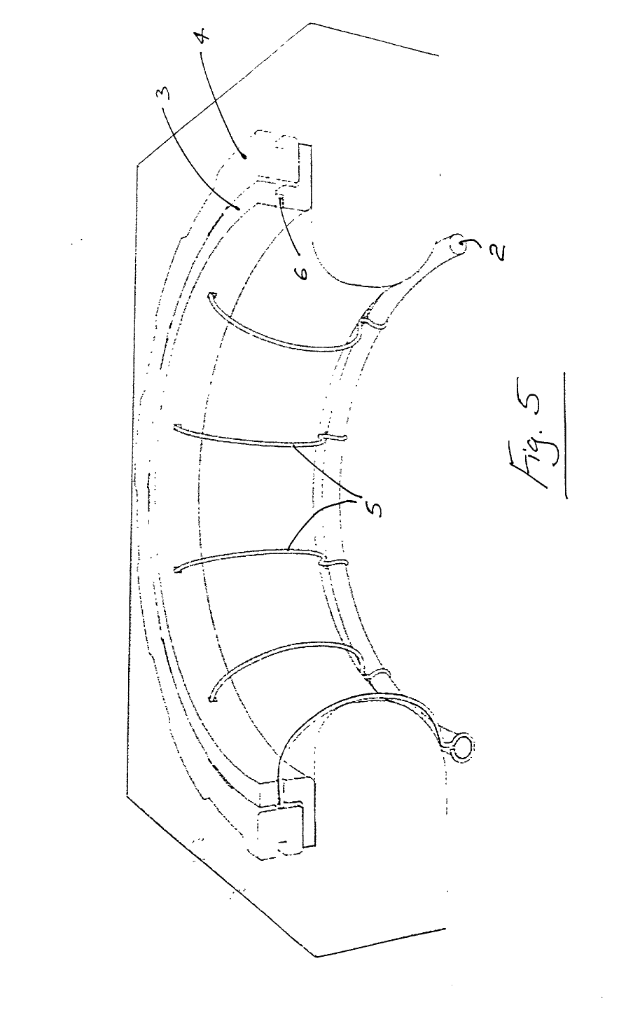

### DETAILED DESCRIPTION

Referring to the drawings there is illustrated a wound retractor device 1 according to the invention. The device 1 consists of a thermoplastic inner O-ring 2 connected to an external ring assembly comprising an inner ring 3 and an outer ring 4 by a plurality of straps 5 of polymeric material. Each strap 5 passes through a guide hole 6 in the inner ring 3 of the external ring assembly to the outer ring 4 to which it is fixed. The rings 3, 4 are slidably rotatable relative to each other. Rotational movement of the outer ring 4 results in shortening of the distance between the external ring assembly and the internal ring 2 by taking up slack in the straps 5 and withdrawing it into the space between the rings 3, 4 of the external ring assembly, effectively shortening the length of and tensioning the straps 5.

The rings 3, 4 include formations 10 to assist in relative rotation of the rings. On tensioning of the straps 5 the rings 3, 4 may be locked by any suitable locking means.

In use, the internal O-ring 2 is inserted through a wound opening such that it is within the space defined by the layers of tissue and is flat against the innermost tissue layer. The external ring assembly should also be flat against the external tissue layer. The outer ring 4 of the external ring assembly is rotated with respect to the inner ring 3 of the external ring assembly to draw the polymer straps 5 up into the space between the inner and outer rings 3, 4 thus shortening the distance between the internal ring 2 and the external ring assembly. This will result in compression of the tissue layers between the internal ring and the external ring assembly. Eventually, the shortening of the straps 5 will displace the margins of the wound laterally and cause retraction of the wound edge and exposure of the inside of the wound site. When adequate retraction has been achieved the inner and outer rings 3, 4 of the external ring assembly are locked in position with respect to each other to maintain retraction.

The device is removed by unlocking the inner and outer rings of the external ring assembly. This allows the polymer straps 5 to effectively lengthen and increases the distance between the internal ring 2 and the external ring assembly. The internal ring 2 may then be removed from the wound opening.

It will be appreciated that the connecting means may be provided by cords or wires, preferably of polymeric material.

The invention is not limited to the embodiments hereinbefore described which may be varied in detail.

1. A surgical wound retractor comprising:
an inner mounting means for insertion into a wound opening to be retained inside the wound opening;
an outer retracting means;
connecting means between the inner mounting means and the outer retracting means;
the outer retracting means being operable to pull the connecting means laterally to retract the wound opening.

2. A retractor as claimed in claim 1 wherein the connecting means comprises a plurality of spaced-apart straps extending between the inner mounting means and the outer retracting means.

3. A retractor as claimed in claim 2 wherein the straps are of pliable material.

4. A retractor as claimed in claim 2 or 3 wherein the straps are of polymeric material.

5. A retractor as claimed in any of claims 1 to 4 wherein the outer retracting means comprises at least two elements, the connecting means being mounted to one of the elements and the elements being movable relative to each other to tension the connecting means.

6. A retractor as claimed in claim 5 wherein the retracting means is a ring assembly comprising a first ring and a second ring, the connector means being mounted to one of the rings and one of the rings being movable relative to the other ring to tension the connecting means.

7. A retractor as claimed in claim 6 wherein the ring assembly comprises an inner ring and an outer ring, the connector means comprising a plurality of straps, each strap being mounted to the outer ring, and the outer ring being rotatable relative to the inner ring to tension the straps.

8. A retractor as claimed in claim 7 wherein the straps extend through guides in the inner ring.

9. A retractor as claimed in any preceding claim wherein the inner mounting means is an O-ring.

10. A retractor as claimed in claim 9 wherein the outer ring assembly has a larger diameter than the diameter of the inner ring means.

11. A retractor as claimed in claim 9 or 10 wherein the inner O-ring is of a flexible material.

12. A retractor substantially as hereinbefore described with reference to the accompanying drawings.
